(19) United States
(12) Patent Application Publication
(10) Pub. No.: US 2011/0106177 A1
(43) Pub. Date: May 5, 2011

(54) ANCHOR FOR USE WITH ORTHOPEDIC SCREW

(76) Inventor: Stephen J. Lewis, Toronto (CA)

(21) Appl. No.: 12/990,128

(22) PCT Filed: Apr. 28, 2009
(86) PCT No.: PCT/CA2009/000570

§ 371 Date: Jan. 18, 2011

Related U.S. Application Data
(60) Provisional application No. 61/048,305, filed on Apr. 28, 2008.

(51) Int. Cl. A61B 17/86; B21H 3/02

(52) U.S. Cl. 606/305; 470/9

(57) ABSTRACT

The invention provides devices and methods for stronger and more stable spinal and other orthopedic implant fixation through the use of an anchor for an orthopedic screw e.g, a pedicle screw. The anchor includes a relatively rigid proximal flange and a distal flexible portion that has an inner surface engageable by the screw shank as it is installed in a patient. For use, the distal portion of the anchor is located in a tract of the patient and the screw then installed through the anchor in the tract. During installation, outwardly-directed radial pressure of the screw shank upon inner surfaces of the flexible distal portion of the anchor can cause enhanced engagement of the bones or body structures by the anchor. The anchor flange can include teeth for engaging bones or other body structures, so as to provide improved support for the orthopedic installation.

### BACKGROUND OF THE INVENTION

The invention relates to spinal and other orthopedic implants. In particular, the invention provides anchors for use with orthopedic screws, methods of installing such an anchor and screw, combinations of the screws and anchors, which are used in conjunction with other devices for installing and supporting spinal and other orthopedic implants in living recipients.

### SUMMARY OF THE INVENTION

In various aspects the invention provides devices and methods for stronger and more stable fixation of spinal and other orthopedic implants through the use of anchors in conjunction with and spinal or other orthopedic implants or assemblages.

In one aspect, the invention is thus an orthopedic fixation system. The system includes an orthopedic screw having a head and a threaded shank. Often the screw is a pedicle screw, and generally the head of the screw includes means for attaching an orthopedic device such as a spinal rod or plate. The system also includes a screw anchor. The anchor includes a flange having a relatively rigid ring. By “relatively rigid” it is meant that the ring will retain its original shape during use. As described further below, the anchor is incorporate into an orthopedic implant in which the screw is installed through the anchor. As part of the installation process, as described further below, the head of the screw comes into abutting contact with the ring of the flange and being relatively rigid will withstand bending or other significant deformation during installation. The ring or flange also has an interior wall defining a generally circular aperture. The shank of the screw passes through the aperture of the ring as part of the orthopedic installation. The anchor also includes a relatively flexible sleeve. The sleeve is affixed at its proximal end to the ring. The proximal end, the portion closer to the exterior of an installation, can also at times be referred to as the “upper” end of the sleeve. In the illustrated embodiment, the sleeve is separately formed from the ring and the parts are affixed to each other during manufacture, but this is not necessarily the case. The sleeve has a wall, the interior surface of which, together with aperture of the flange defines a passage for receipt of the shank of the screw therethrough. In the illustrated embodiment, the sleeve is a single sheet of metal, but again this is not necessarily the case. The sleeve is relatively flexible, relative to the ring or flange of the anchor. The distal end of the sleeve has a reduced cross dimension relative to the cross dimension of the shank of the screw when installed therein and the wall of the sleeve has a discontinuity at least the distal end of the sleeve, such that as a leading end of the shank of the screw travels through the passage from the proximal end to the distal end of the sleeve and the shank engages the interior surface of the sleeve wall the discontinuity permits radial expansion as by e.g., flexing of the sleeve. By “relatively flexible” the sleeve is capable of bending during installation. That is, as the screw is threaded into place within a tract of the bone, the flange by force of the screw shank thereagainst expands and becomes lodged into place between the screw shank and wall of the bone tract. The “discontinuity” in the wall of the illustrated embodiment is provided by a the metal wall being a rolled into a somewhat frustoconical conical shape in which longitudinal portions metal overlap with each other. The overlapping portions are not fastened to each other and so are free to move relative to each other as the shank moves through passage and mechanically forces the wall radially outwardly.

So, in installed condition, the base of the screw head and upper surface the ring of the illustrated embodiment are in abutment with each other. The sleeve extends along the proximal end of the screw shank. According to various embodiments, The sleeve extends at least 10% of the shank length; or at least 15% of the shank length; or at least 20% of the shank length; or at least 25% of the shank length; or at least 30% of the shank length; or at least 35% of the shank length; or at least 40% of the shank length; or at least 45% of the shank length; or at least 50% of the shank length; or at least 55% of the shank length; or at least 65% of the shank length; or at least 75% of the shank length; or at least 80% of the shank length; or at least 85% of the shank length; or at least 90% of the shank length. The length of the shank extends from the base of the screw head to the tip of the shank, so in the case where the screw shank is 50 mm and the sleeve is 10 mm in length, the sleeve extends about 20% along the length of the screw shank.

In installed condition, the sleeve can extends up to 100% of the shank length; or up to 95% of the shank length; or up to 90% of the shank length; or up to 85% of the shank length; or up to 80% of the shank length; or up to 75% of the shank length; or up to 70% of the shank length; or up to 65% of the shank length; or up to 60% of the shank length; or up to 55% of the shank length; or up to 50% of the shank length; or up to 45% of the shank length; or up to 40% of the shank length; or up to 35% of the shank length; or up to 30% of the shank length; or up to 25% of the shank length; or up to 20% of the shank length; or up to 15% of the shank length.

According to particular embodiments, in installed condition, the sleeve extends between about 10% and 100% of the shank length; or between about 10% and 85% of the shank length; or between about 10% and 75% of the shank length; or between about 10% and 65% of the shank length; or between about 15% and 65% of the shank length; or between about 15% and 50% of the shank length; or between about 15% and 40% of the shank length; or between about 15% and 30% of the shank length.

According to other embodiments, the sleeve extends about 10% of the shank length; or about 15% of the shank length; or about 20% of the shank length; or about 25% of the shank length; or about 30% of the shank length; or about 35% of the shank length; or about 40% of the shank length; or about 45% of the shank length; or about 50% of the shank length; or about 55% of the shank length; or about 60% of the shank length; or about 65% of the shank length; or about 70% of the shank length; or about 75% of the shank length; or about 80% of the shank length; or about 85% of the shank length.

In the illustrated embodiment, the sleeve wall is metal obtained by rolling a precut sheet into the desired configuration. The discontinuity is provided by overlapping edges of the metal sheet once rolled to form the sleeve.

The sleeve wall of certain embodiments is provided by a sheet of curved metal having first and second edges which are generally parallel to a central axis of the passage through the anchor. The edges overlap with each other both before and after the shank travels through the passage, to provide a continuously surrounding wall, when the system is installed in an orthopedic implant, the surrounding surface being for contact with surrounding bone matter. Here, “continuously surrounding surface” means that the wall completely surrounds the anchor shank along the length of the sleeve, even though there is a discontinuity in the sleeve wall in this embodiment provided by overlapping edges of sleeve metal.

The anchor flange and sleeve are preferably made of the same metal and the screw and anchor are also preferably made of the same metal as each other. Suitable metals are stainless steel or titanium.

In an anchor in which the sleeve is formed of a single piece of sheet metal, the metal sheet would typically have a substantially constant thickness, between about 0.002 inches (0.0508 mm) and about 0.006 inches (0.1524 mm), preferably between about 0.003 inches (0.0762 mm) and about 0.005 inches ( )0.127 mm), more preferably about 0.004 inches (0.1016 mm).

A screw commonly used as part of the fixation system has a head that is generally convex at its base i.e., at its distal end, and the upper surface of the ring of the flange is generally concave such that in installed condition, a distal portion of the head is received into the ring.

According to one embodiment, the proximal end of the sleeve is a metal sheet affixed to the ring by laser welding.

The sheet metal from which the sleeve is made, in the illustrated embodiment, is precut and once formed into the sleeve defines tabs along the proximal edge of the sleeve. The tabs are preformed in the metal sheet and bent to depend radially outwardly from the sleeve wall to abut the upper surface of the ring. The tabs and ring are laser welded to each other.

The shank of the orthopedic screw can have any desired length. Most often being a pedicle screw, the screw shank has a length of between 25 mm and 60 mm, or the shank as a length of between 25 mm and 60 mm, or the shank has a length of about 25 mm, 30 mm, 35 mm, 40 mm, 45 mm, 50 mm, 55 mm or 60 mm.

The shank of the screw typically has an outer diameter of between about 4.5 mm and 8.5 mm, or is about 4.5 mm, 5.0 mm, 5.5 mm, 6.0 mm, 6.5 mm, 7.0 mm, 7.5 mm, 8.0 mm or 8.5 mm.

According to an aspect of the invention, the aperture of the flange is sufficiently large to permit the shank of the screw to freely pass therethrough.

According to another aspect the ring aperture and screw shank are dimensioned such that, in installed condition, at least 60% of the cross sectional area of the passage through the aperture is occupied by the shank, or at 70% of the cross sectional area of the passage through the aperture is occupied by the shank, or at least 80% of the cross sectional area of the passage through the aperture is occupied by the shank.

The ring aperture and screw shank can be dimensioned such that, in installed condition, no more than 90% of the cross sectional area of the passage through the aperture is occupied by the shank.

In illustrated embodiments, the ring aperture and screw shank are dimensioned such that, nominal calculations indicate that between about 72% and 82% of the cross sectional area of the passage through the flange aperture is occupied by the shank.

In one aspect, the sleeve of the anchor has a length of at least about 10 mm.

According to another aspect, the sleeve of the anchor has a length of no more than about 45 mm, more preferably no more than about 35 mm.

The sleeve can have a length of from about 10 mm to about 45 mm, or from about 10 mm to about 40 mm, or from about 10 mm to about 35 mm, or from about 10 mm to about 30 mm, or from about 10 mm to about 25 mm, or from about 10 mm to about 20 mm, or from about 15 mm to about 45 mm, or from about 15 mm to about 40 mm, or from about 15 mm to about 35 mm, or from about 15 mm to about 30 mm, or from about 15 mm to about 30 mm, or from about 15 mm to about 25 mm, or from about 15 mm to about 20 mm, or from about 20 mm to about 45 mm, or from about 20 mm to about 40 mm, or from about 20 mm to about 35 mm, or from about 20 mm to about 30 mm, or from about 20 mm to about 25 mm, or from about 25 mm to about 45 mm, or from about 25 mm to about 40 mm, or from about 25 mm to about 35 mm, or from about 25 mm to about 30 mm, or from about 30 mm to about 45 mm, or from about 30 mm to about 40 mm, or from about 30 mm to about 35 mm, or from about 40 mm to about 45 mm, or the sleeve can have a length of about 10 mm, or about 15 mm, or about 20 mm, or about 25 mm, or about 30 mm, or about 35 mm.

According to another aspect, in installed condition, the sleeve of the anchor has a length relative to the length of the screw shank such that at least 80% of the full length of the shank is exposed, or at least 70% of the full length of the shank is exposed, or at least about 60% of the full length of the shank is exposed, or at least about 50% of the full length of the shank is exposed, or at least about 40% of the full length of the shank is exposed, or at least about 30% of the full length of the shank is exposed, or at least about 20% of the full length of the shank is exposed, or at least about 10% of the full length of the shank is exposed. In this context, “exposed” means that once the screw and anchor are fully implanted together in a bone, the portion of the screw shank that is not surrounded by the sleeve is the exposed portion of the shank, this being the distal end of the shank in the illustrated embodiments.

In another aspect, in installed condition, the sleeve of the anchor has a length relative to the length of the screw shank such that up to about 90% of the full length of the shank is exposed, or up to about 80% of the full length of the shank is exposed, or up to about 70% of the full length of the shank is exposed, or up to about 60% of the full length of the shank is exposed, or up to about 50% of the full length of the shank is exposed, or up to about 40% of the full length of the shank is exposed, or up to about 30% of the full length of the shank is exposed, or up to about 20% of the full length of the shank is exposed, or up to about 10% of the full length of the shank is exposed.

In yet another aspect, in installed condition, the sleeve of the anchor has a length relative to the length of the screw shank such that between about 0% and 80% of the full length of the shank is exposed, or between about 10% and 80% of the full length of the shank is exposed, or between about 20% and 80% of the full length of the shank is exposed, or between about 30% and 80% of the full length of the shank is exposed, or between about 40% and 80% of the full length of the shank is exposed, or between about 50% and 80% of the full length of the shank is exposed, or between about 60% and 80% of the full length of the shank is exposed, or between about 0% and 70% of the full length of the shank is exposed, or between about 10% and 70% of the full length of the shank is exposed, or between about 20% and 70% of the full length of the shank is exposed, or between about 30% and 70% of the full length of the shank is exposed, or between about 40% and 70% of the full length of the shank is exposed, or between about 50% and 70% of the full length of the shank is exposed, or between about 0% and 60% of the full length of the shank is exposed, or between about 10% and 60% of the full length of the shank is exposed, or between about 20% and 60% of the full length of the shank is exposed, or between about 30% and 60% of the full length of the shank is exposed, or between about 40% and 60% of the full length of the shank is exposed, or about 10% of the full length of the shank is exposed, or about 20% of the full length of the shank is exposed, or about 30% of the full length of the shank is exposed, or about 40% of the full length of the shank is exposed, or about 50% of the full length of the shank is exposed, or about 60% of the full length of the shank is exposed, or about 70% of the full length of the shank is exposed, or about 80% of the full length of the shank is exposed.

According to particular aspect of the invention, the flange of the sleeve includes at least one tooth on a distal side of the flange ring for engaging bone in abutment with the distal side of the ring when the device is installed in a patient. In the illustrated embodiments, wherein the tooth and ring are of unitary construction.

It is advantageous to include a plurality of such teeth in which at least two of the teeth are angularly pointed towards each other such that when the device is installed in a patient both of the teeth are embedded in bone such that the anchor is fixed against rotation in both the counterclockwise direction and the clockwise direction with respect to a central axis of the aperture of the anchor.

To assist in installation of the screw and anchor, the flange can include means for fixing the anchor against rotation during installation of the screw. According to one aspect, the means for fixing the anchor against rotation is a depression in a surface of the ring for receipt of a tool therein. In another aspect, the means for fixing the anchor against rotation comprises a raised abutment which protrudes from the flange. Either the depression or abutment, or combination thereof is located suitably for its intended purpose during installation and may be situated on the proximal outwardly facing surface of the flange and/or the curved side surface of the flange.

According to another aspect, the invention is an anchor for use in an orthopedic fixation system that includes an orthopedic screw. The anchor includes a flange having a relatively rigid ring having an interior wall having a generally circular aperture. The anchor has a relatively flexible sleeve affixed at its proximal end to the ring. The sleeve has an interior wall which together with the aperture of the flange defines a passage for receipt of the shank of the screw therethrough. The distal end of the sleeve has a reduced cross dimension relative to the cross dimension of the shank of the screw when installed therein and the wall of the sleeve has a discontinuity at least the distal end of the sleeve, such that as a leading end of the shank of the screw travels through the passage from the proximal end to the distal end of the sleeve and the shank engages the interior wall of the sleeve the discontinuity permits outward radial expansion of the sleeve.

The anchor can be for use with a screw having shank that is at least about 25 mm in length and the sleeve has a length of at least about 10 mm.

The anchor can be for use with a screw having shank that is up to about 50 mm in length and the sleeve has a length of up to about 50 mm.

In further aspects the invention provides methods for the placement of spinal or other orthopedic screws and anchors, and implant devices supported by such screws and anchors, in anterior and/or posterior portions of vertebrae of living subjects.

For example, in various embodiments such methods provide for placement of one or more spinal implants in a living subject by exposing anterior aspects of a plurality of the subject's vertebrae, establishing at least one tract for a vertebral body screw in each of a plurality of the exposed vertebrae (which, again, may or may not be contiguous or consecutive); placing an anchor into each established tract; installing a vertebral body screw into each anchor, thereby causing the anchor to expand for firmer engagement of the corresponding vertebra; securing a spinal implant, such as a rod or plate, to the screw; and optionally providing closure for the exposed vertebrae.

In further embodiments of such methods, the invention provides for placing one or more spinal implants in a living subject by locating at least one pedicle on each of a plurality of vertebrae of the subject and establishing in each located pedicle a tract for insertion of a pedicle screw; placing an anchor into each established tract; installing a vertebral body screw into each anchor, thereby causing the anchor to expand for firmer engagement of the corresponding vertebra; and securing a spinal implant to the screw.

In various embodiments of methods according to such aspects of the invention, location of vertebrae, establishment of tracts for the insertion of screws and anchors, and/or insertion of screws and anchors can be performed using percutaneous or other minimally-invasive procedures.

In various embodiments, methods according to the invention can further comprise configuring implants, such as rods or plates, into one or more desired shapes, either before or after attachment to implanted screws.

Such methods can further comprise the use of anchors having toothed flanges, including installation of such anchors such that at least one of the teeth provided on such flanges engages bone, so as to support the corresponding anchor(s) and thereby enhance support of any screws and/or devices implanted in association with the anchor(s).

Additional aspects of the invention will be apparent in view of the description which follows.

### BRIEF DESCRIPTION OF THE FIGURES

The invention is illustrated in the figures of the accompanying drawings, which are meant to be exemplary and not limiting, and in which like references are intended to refer to like or corresponding parts.

FIG. 1A is a schematic diagram of an embodiment anchor of the invention shown with a screw.

FIG. 1B is a sectional of the FIG. 1A anchor taken along line 1B-1B of FIG. 1A.

FIG. 2A is a drawing of a metal sheet from which a sleeve can be manufactured.

FIG. 2B shows a top view (upper) and a side view (lower) of a flange ring.

FIG. 3 shows a detail of the head of a screw installed in an anchor.

FIGS. 4A-4E illustrate an anchor having a toothed flange.

FIG. 5 is a schematic of an anchor having a variant sleeve.

FIGS. 6 to 8 are schematic diagrams of placement of screw and anchor sets in vertebrae in accordance with the invention.

FIGS. 9 to 11 are schematic diagrams illustrating problems preventable through use of the invention.

FIG. 12 is a schematic diagram of placement of screw and anchor sets in vertebrae in accordance with the invention.

### DETAILED DESCRIPTION OF EMBODIMENTS

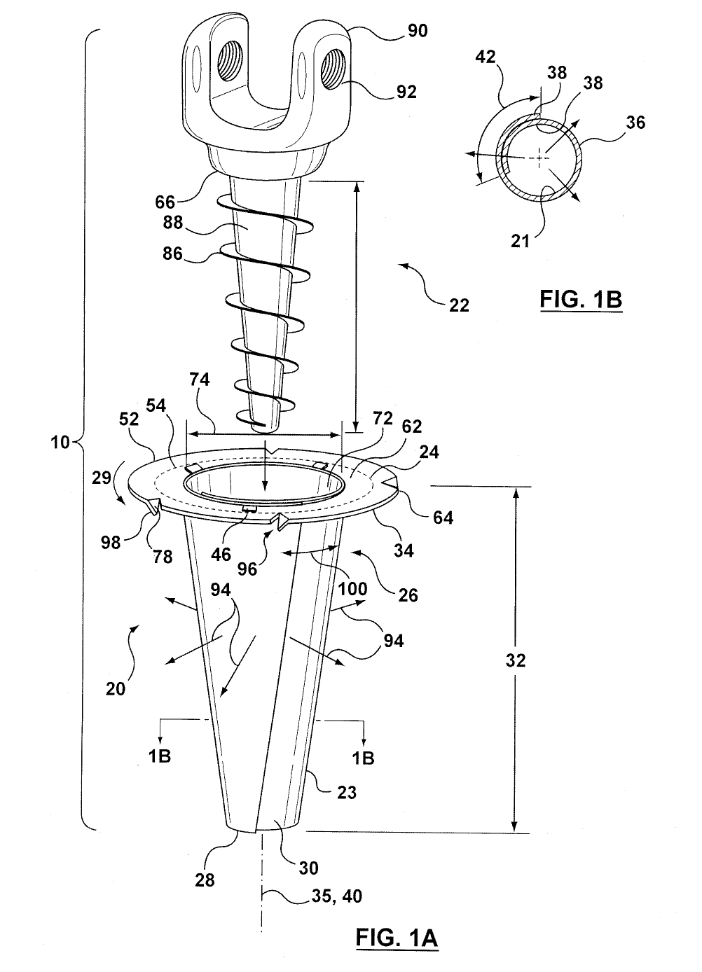

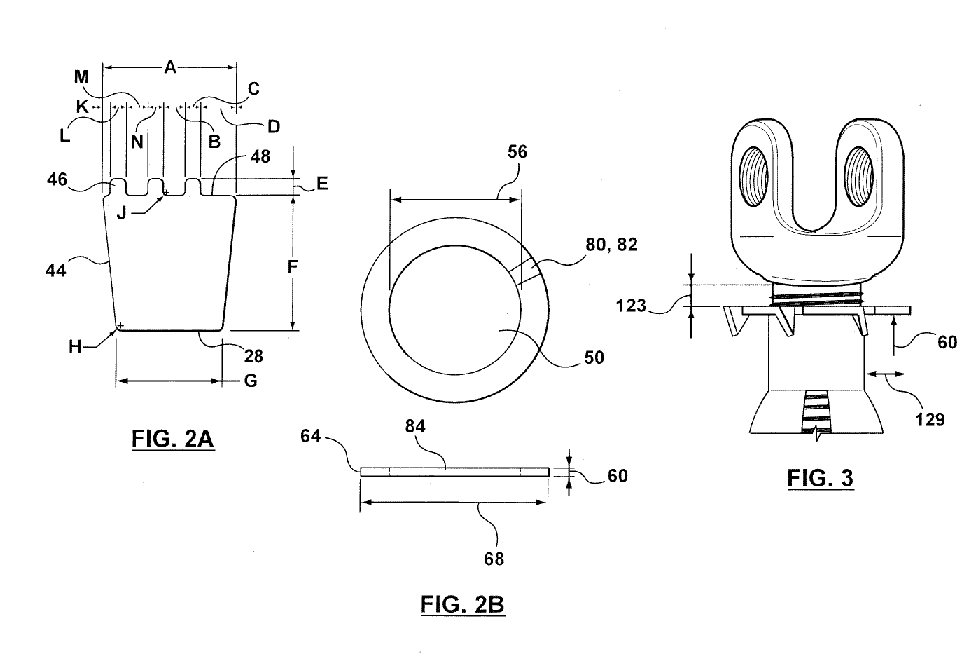

Turning to the drawings, an anchor-screw combination 10 made up of a first embodiment anchor 20 and screw 22 is illustrated in FIG. 1A. Anchor 20 includes sleeve 23 and flange 24, the flange being at the proximal end 26 of the anchor and the leading edge 28 of the sleeve being at the distal end 30 of anchor 20.

The sleeve is formed of any suitable material i.e., physiologically compatible with required physical characteristics. Stainless steel suitable for surgical purposes, “surgical steel” or titanium are most likely preferred. This is because orthopedic screws are generally made of surgical steel or titanium and since one would generally match the anchor material with that of the orthopedic screw with which it is to be used. The length 32 of the sleeve (as measured from leading edge 28 to base or underside surface 34 of the flange) can be of any convenient length, most likely being at least 10 mm and rarely more than about 50 mm. A preferred length is either about 10 mm or about 25 mm, advantages to each of these lengths being described further below.

A titanium or surgical steel sleeve is typically formed from a sheet of the metal which is of a relatively constant thickness 36 (FIG. 1B) of about 0.004 inches (0.1016 mm), but might be slightly less or slightly more than this, possibly in the range of 0.002 inches (0.0508 mm) to about 0.006 inches (0.1524 mm), although a thickness of 0.002 inches (0.0762 mm) was found not to work as well as a thickness of 0.004 inches, being more flimsy and making it more difficult to install. A sleeve that is too thick requires a typically undesirable reduction in the diameter of the screw to be used with anchor.

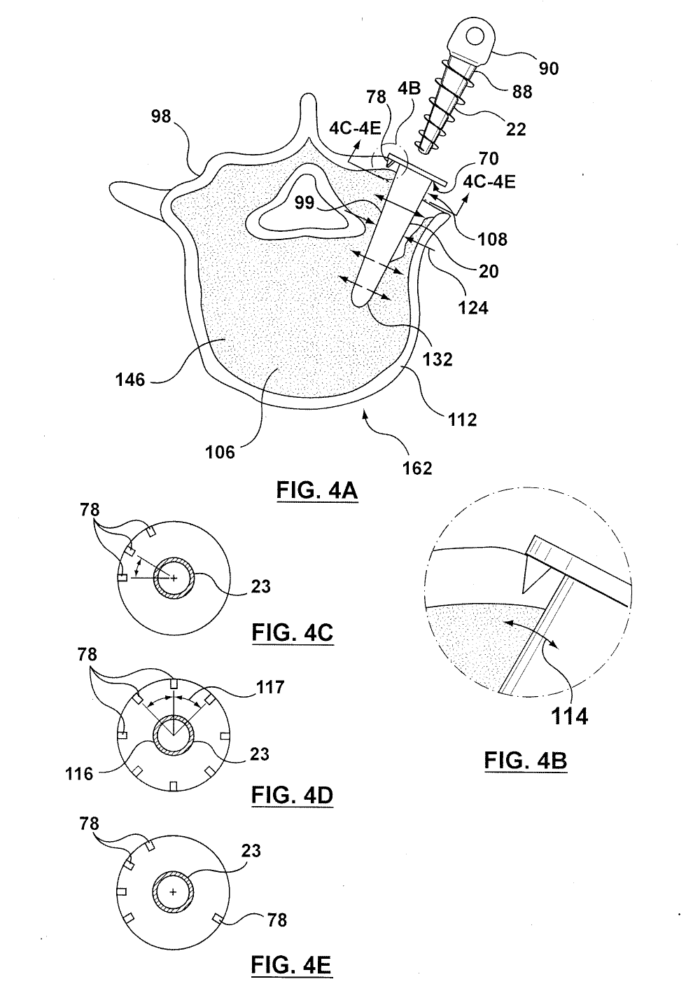

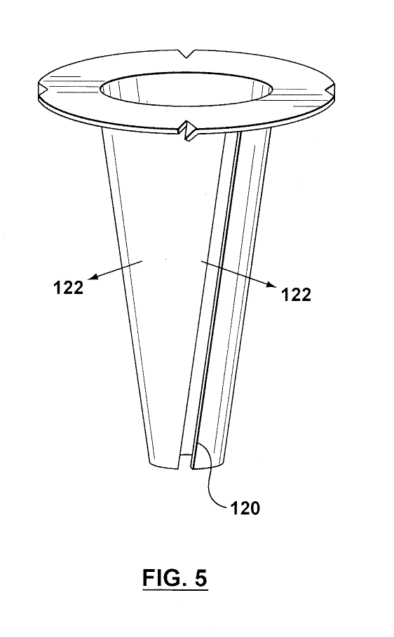

The metal sheet from which the sleeve is obtained is dimensioned such that the edge portions of the metal that are somewhat parallel to the central axis 40 of the anchor overlap with each other. Dimensions of sheet from which an anchor sleeve of the invention was formed is shown in FIG. 2A, and the degree of overlap of the formed anchor can be seen in the end view of the distal end in FIG. 1B in which the degree of overlap 42 at the distal end is visible. The degree of overlap can be measured as the percentage of the entire circumference of the sleeve wall in which overlap occurs. For illustrative purposes, overlap of 25% at the distal end of the sleeve is shown in FIG. 2A.

The dimensions of a specific embodiment are shown in FIG. 2A:

| Dimension Label | Value - Inches (mm) |
|---|---|
| A | 0.984 (25.0) |
| B | 0.158 (4.0) |
| C | 0.118 (3.0) |
| D | 0.263 (6.7) |
| E | 0.118 (3.0) |
| F | 0.984 (25.0) |
| G | 0.787 (20.0) |
| H | Radius = 0.035 (0.9) |
| J | Radius = 0.020 (0.5) |
| K | 0.052 (1.3) |
| L | 0.118 (3.0) |
| M | 0.158 (4.0) |
| N | 0.118 (3.0) |

As mentioned previously, thickness 36 of the illustrated sheet is 0.004 inches.

Sheet 44 from which the sleeve is formed is shaped, as by cutting, to obtain a plurality of tabs 46 along the proximal edge 48 of the sheet. The metal is rolled into the shape of the sleeve and the tabs are bent radially outwardly. Leading edge 28 of the sleeve is passed through the aperture 50 of the ring 52 from proximal (outer) side 54 of the ring which forms the flange in the anchor. The inside surfaces of the tabs are brought into contact with the proximal (outer) surface of the ring and suitably fastened to the ring by e.g., laser welding to form the anchor. The illustrated embodiment has three tabs 46 spaced from each other so that they are approximately evenly spaced about the ring in the assembled anchor.

The sleeve is formed to be in an initially tapered (slightly conical) shape. This permits convenient installation within a bone cavity in which it is to be installed.

Metal ring 52 forming flange 24 is of the same material as the sleeve. Central aperture 50 of the ring of the first embodiment has inner diameter 56 that is 1 mm greater than the diameter of the shank of the screw with which it is to be used. This has been found satisfactory, sufficiently accommodating the reduction in aperture size with installation of the sleeve within the ring aperture. In the case of the illustrated embodiment, the anchor is for use with a pedicle screw having a shank having an outer diameter of about 5.5 mm and inner diameter 56 of the ring (prior to installation of the sleeve) is 6.5 mm. The inner diameter of the ring aperture, once the sleeve is installed, thus suitably matches the maximum outer diameter of the screw shank, but could be slightly greater than the shank. So one can thus see that the passage defined by flange aperture 72 in the anchor has a nominal diameter 74 that is determined by subtracting from the inner diameter of the ring 76 a length equal to twice the thickness 36 of the sheet from which the sleeve is manufactured. Specifically in the illustrated embodiment, the calculated diameter of the flange aperture is 74 is 6.5−(2×0.1016)=6.3 mm.

Thickness 60 of the ring can be of any suitable thickness, a thickness of between about 1 to 6 mm being suitable for many purposes. The illustrated embodiment has a thickness of 2 mm at the outer wall of the ring. Distal surface 34 (underside) of the ring generally runs in a plane perpendicular to the central axis 35 of the sleeve. The proximal (outer) surface of the ring has a surface 62 which cants toward i.e., in t the direction of the underside of the ring as it runs radially inwardly from the outer circular wall 64 towards central aperture 50. Surface 62 of the side of the ring of the illustrated embodiment is additionally concave and shaped to generally match or complement the convex underside 66 of the orthopedic screw to be installed with the anchor.

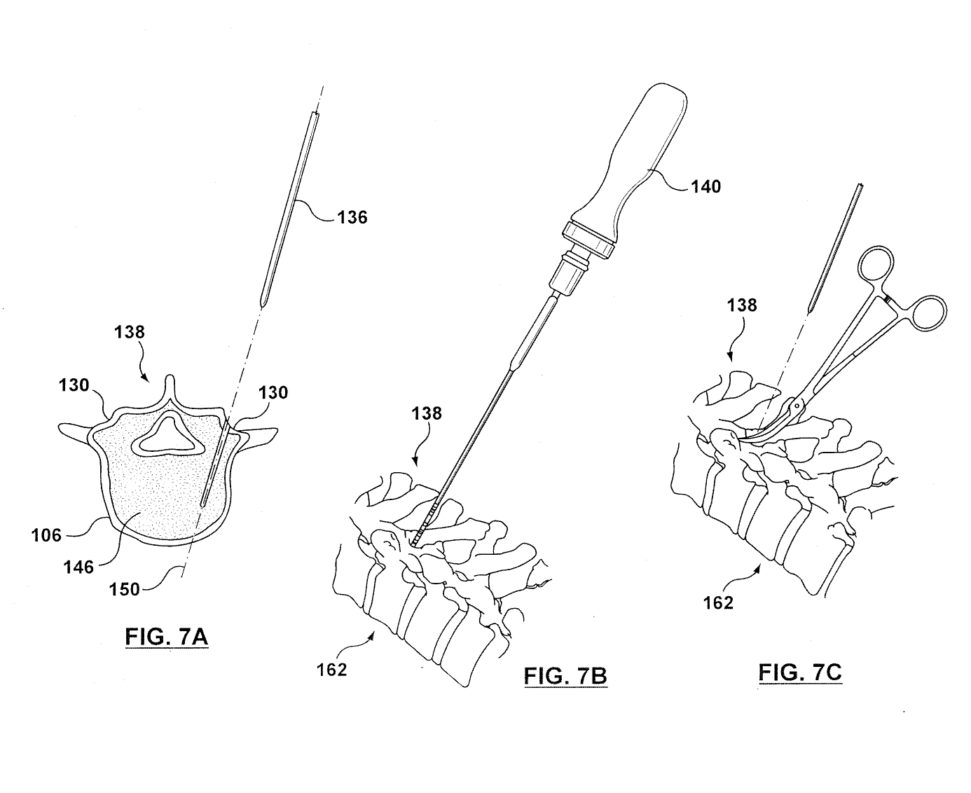

The outer diameter 68 of the ring from which the flange is formed can be determined by the extent of overhang 70 the flange is desired to have with respect to the sleeve. See FIG. 4A. Preferably, underside 34 of the ring has teeth 78 formed integrally with it. The ring of the illustrated embodiment is manufactured by conventional computer controlled numeric cutting and teeth of appropriate size and configuration can be conveniently cut into the underside of the ring. As described further below, to the extent that the teeth engage the bone, and the anchor and screw engage each other, possible twisting and loosening of the screw with respect to the bone as through stress placed on the orthopedic installation is reduced thereby improving the stability of the installation. To the extent that the environment of an installation permits (bone surface configuration, etc.) it is preferable to install the anchor such that the teeth become embedded in the bone. It is thus thought that it would be preferred for there to be multiple teeth e.g. at least two or more teeth and that teeth 78 should be angled oppositely (in the angular sense) from each other such that the installed anchor resists rotation in both clockwise and anticlockwise directions. The teeth should be situated on the ring such that in use they are located in the proximal and medial portion of the pedicle start point to anchor in the stronger bone but to avoid the exiting nerve root which passes around the medial and inferior aspect of the pedicle.

As mentioned above, the illustrated embodiment can be of any desired length as suitable for a particular installation. It is preferred in practice for a pedicle or other orthopedic screw to be installed within a wholly intraosseous tract. This provides for a more resilient installation than one in which is not entirely intraosseous. In such an entirely intraosseous installation, it is thought that an anchor having a sleeve which extends only partially along the screw shank is preferred. In the context of an installation in a vertebra a sleeve having a length of about 10 mm can often be suitable. If an anchor having a 10 mm length sleeve is used with a pedicle screw having a 25 mm shank, the sleeve extends along approximately 40% of the proximal portion of the shank. A 10 mm length sleeve of an anchor used with a pedicle screw having a 50 mm shank would extend along approximately 20% of the proximal portion of the shank.

A less ideal situation arises when the tract in which the screw and anchor are to be installed is not entirely intraosseous, as illustrated in FIG. 4A. This can occur, for example, if part of the bone has been removed, broken, or the requirements of the installation are such that the screw is directed through spaced apart bone portions i.e., as by an in-out-in insertion technique. In such an installation, it is thought that a sleeve of sufficient length to cross the extraosseous gap is preferred. In such case, an anchor having a sleeve of about 2.5 cm in length will often be suitable.

The anchor can also include means for engaging 80 an installation tool. The means 80 would permit fixing the anchor against rotation as the screw is rotated within the sleeve during installation. The fixation means 80 would most likely be associated with the flange of the anchor for example, radial groove 82. The tool, having rotary driving means for installation of the screw, could include means for engaging groove 82 so that the anchor is fixed against rotation with respect to the screw during installation, or a separate tool could be used to fix the anchor against rotation. Ultimately, engagement of flange teeth 78 with surrounding bone affixes the anchor against rotation. The installation tool may or may not be mechanized, and the tool for engaging means 80 can be a hand held tool that is separate from the tool used for installation of the screw within the bone tract.

Dimensions relevant to the user, e.g., an orthopedic surgeon can be conveniently cut, etched or stamped onto a visible portion of the anchor. For example the nominal inner diameter of the ring aperture could be located on outer curved surface 84 of the ring e.g., 4.5 mm. Likewise the nominal length of the sleeve as it extends below the underside of the ring could be located on the sleeve itself e.g. 10 mm. It may be preferable for both dimensions to be located on ring surface 84 e.g. “4.5 mm dia×10 mm” or on the sleeve. The choice of length and diameter of screw to be installed at a particular location within the context of an orthopedic implant e.g., a spinal rod can vary. Such markings may be important to the user where the screws and anchors are provided as separate items in order to aid the surgeon in selecting the appropriate anchor once the length and diameter of screw is selected for implantation.

The schematic diagram the FIG. 1 embodiment illustrates screw 22, which can be a spinal screw such as a vertebral body screw or a pedicle screw, or any other type of orthopedic screw, includes thread 86 on shank 88 and integral, removable, or partially removable head 90, which can include any means 92 suitable for use in attaching or otherwise supporting an implant device such as a spinal implant rod or plate. Screw heads 90 suitable for use with the invention can comprise, for example, screw caps and crimping devices such as those provided by K2M, Synthes, Medtronic, Biomet, Zimmer, Stryker, Johnson & Johnson-Depuy Spine, Ausculap, Spine Vision.

Anchor 20 includes sleeve 24, which is configured to receive shank 88 of screw 22 and, upon insertion, as for example by driving, of screw 22 into sleeve 23, to expand radially in the direction(s) of arrows 94, so as to cause anchor 20 to engage bone or other structure of a body into which anchor 20 has been inserted in such a way as to more stably support screw 22 and any devices attached thereto, including for example spinal or other implant structures.

In the embodiment shown in FIG. 1, anchor 20 comprises flange 24, which includes a plurality of teeth 78 adapted for engagement with bone or other structures of a body into which the anchor 20 is implanted, as for example by digging into or piercing a hard bony covering or other surface of a vertebra. By engaging a bone or other structure in such fashion, teeth 78 can provide improved lateral support, in the sense provided by arrows 99 (FIG. 4A), and/or rotational support, in the sense provided by arrows 100 (FIG. 1), for anchor 20, and therefore any screw 22 installed therein, and any implant devices attached to the screw 22.

In the embodiment shown in FIGS. 1 and 2, teeth 23 of flange 22 can be formed by laser cutting or by other means of cutting and bending the material of which flange 24 is fabricated. As will be understood by those skilled in the relevant arts, teeth 78 can be provided by any means consistent with the purposes disclosed herein, including for example by integral forming or by attachment of separate structures to flange 24 or other portions of anchor 20. As will be further appreciated by those skilled in the relevant arts, teeth 78 of anchors 20 can further be provided in various lengths and configurations, in order to accomplish various purposes or achieve various advantages that arise under diverse conditions in spinal and other implant procedures. For example, teeth 78 can be of length sufficient to pierce through a bony wall or lamination of the outer surface of a vertebra, and/or can be oriented with angular offsets or other biases 96, 98, to counteract rotation of the anchor 20 in one or both directions 100, 102, such as might otherwise occur during installation and/or removal of set 10 and/or screw 22, or removal of a removable screw head 90, such as is commonly provided on some types of orthopedic screws.

The provision and use of teeth 78 can be of particular advantage in an orthopedic implant where for example a fracture has occurred in bone, or where an anchor 20 must otherwise be installed where a portion of flange 24 and/or sleeve 23 are exposed, or otherwise do not engage a bone or other structure in which they are installed. For example, where a portion of a pedicle and/or transverse process 104 has broken off a vertebra 106, as shown in FIG. 4, an anchor can be installed as shown, with a portion 108 of sleeve 23 and flange 24 exposed, and other portions engaging vertebra 106 proximate to the missing portion 110, in such fashion that one or more teeth 78 of flange 24 pierce or otherwise engage the outer surface or wall 112 of vertebra 106, as shown in Detail 3A. By engaging the outer surface or wall 112 or other portion of vertebra 106, tooth or teeth 78 can support anchor 20 against movement in, for example, lateral direction 114, so that the anchor is less likely to work loose over time. The tooth or teeth will be of appropriate length to engage the structural bone without injuring the adjacent neural or vascular structures.

As noted, teeth 78, if provided, may be provided singly or in multiples. Where multiple teeth 78 are provided, they may be provided at regular and/or irregular intervals, and in symmetrical or asymmetrical patterns, around all or any desired portion(s) of the periphery of flange 24 and/or other portions of proximal end 26 of anchor 20. For example, as shown in FIGS. 4B to 4D, a plurality of teeth 78 may be provided in one or more groupings on one side of flange 24, through arc(s) 116 of any suitable or otherwise desired angle(s), and with any suitable or otherwise desired spacing(s). For example, when a anchor 20 is to be installed within a broken vertebra, as shown in FIG. 4, and relatively solid bone is available for engagement of teeth 78 on only one side of the fracture, it may be desirable to provide teeth 78 only on that portion of flange 22 intended to be disposed in close proximity to such relatively solid bone, as shown in FIG. 4B. Where relatively solid bone structure is expected to be available around all or a relatively larger portion of the periphery of the anchor flange 24, teeth 78 may be provided around the entire circumference of the periphery, as shown in FIG. 4C. And where at least some relatively solid structure suitable for supporting teeth 78 is available on both sides of a fracture or discontinuity, teeth 78 may be provided on both sides of the periphery, as shown in FIG. 4D.

As will be understood, where multiple teeth 78 are to be provided on anchor 20, they may be provided in any suitable intervals, as determined by factors such as practicality of manufacturing and installation, including materials or teeth-forming processes, and/or the geometry, strength, and other characteristics of the bone or other structure by which it is expected that teeth 78 will be engaged. It may be convenient, for example, to provide teeth 78 at regular or progressive angular spacing(s) 116, 118 of 5°, 10°, 15°, 20°, 30°, 45°, 60°, 90°, or other degrees, and as mentioned above teeth can be angled in opposite angular directions so as to create resistance to rotation in both rotational directions of an implanted screw and anchor set.

Anchor 20 and screw 22 may be formed of any materials suitable for the purposes described herein, including for example surgical steel, other metals, plastic(s), and/or biodegradable materials in any suitable or desired combinations. As will be understood by those skilled in the relevant arts, it can be advantageous to select the material or materials used in fabrication of screw 22 and anchor 20 such that corrosion or other undesirable interactions do not occur during prolonged exposure to the types of environments in which such devices are expected to serve.

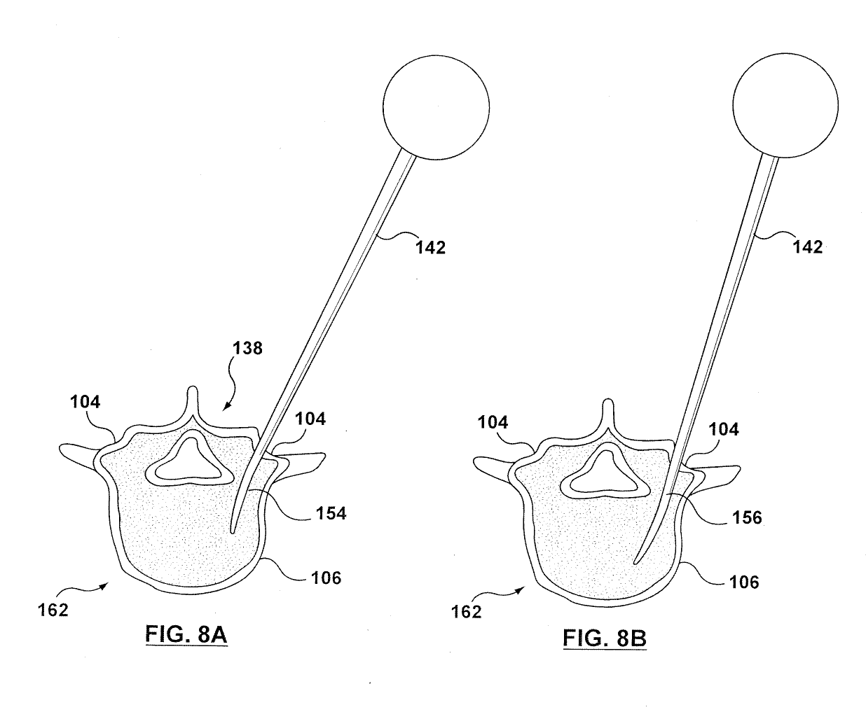

Anchor 20 can be formed in any suitable manner. In some embodiments anchor 20 can be formed economically and effectively by rolling of sheet material of suitable gage, such as a sheet of surgical stainless steel of a desired thickness, into a conical or other desirable shape. Anchor 20 can also be formed by other processes, including for example extrusion and deformation. It may be advantageous to provide other features, such as slit 120 of FIG. 5 or other features which can be provided to enhance deformation and radial expansion of sleeve 23 of anchor 20. As described above anchor 20 of the FIG. 1 embodiment has the sleeve provided in a rolled configuration such as that shown in FIG. 1B, such that radial expansion 122 of the sleeve 23 is possible without slit(s) 120, through ‘unrolling’ action in helical overlap portions 38 of sleeve 23 caused by installation of a screw 22.

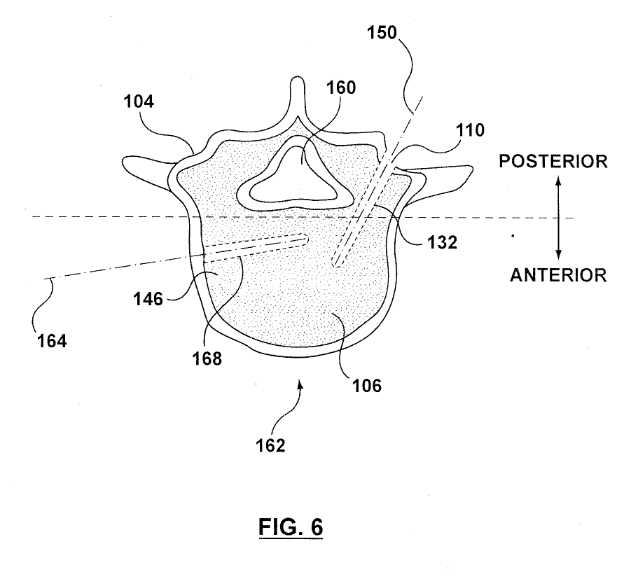

It may be advantageous to provide slits 120 or other mechanisms configured to provide for a predetermined amount of radial expansion 94, 122 dependent upon a known number of turns of a screw 22 after it has been seated in an anchor 20; and to provide for a desired gap 123 (FIG. 3), such as 0.5 mm or less, between the lower surface of the screw head and the upper surface of anchor flange 24. As will be understood by those skilled in the relevant arts, such predetermined amounts of radial expansion may be provided by taking into account such factors as the dimension(s) of anchors 20 and screws 22, including any taper(s) in anchors 20 and thread geometries of screws 22. It is expected that in at least some embodiments of the invention essentially full contact between the screw head and the top of the sleeve will provide a good, practical working configuration.

As will be understood by those skilled in the relevant arts, it may be advantageous to form anchor 20 and in particular sleeve 23 in a generally conical shape so as, for example, to improve its qualities for being implanted in bones or other structures, and, depending upon the desired configuration of screw 22, for mating with or otherwise receiving shank 88 of screw 22 so as to enhance radial expansion of the anchor 20 as the screw 22 is installed.

For anchors intended for spinal implant applications in humans, it has been determined that exemplary dimensions for anchors 20 can comprise flange thickness 81 (see e.g. FIG. 3) of about 1 to 6 mm; maximum sleeve inner diameters of 4.5 to 8.5 mm, including particularly 6.5-7.5 mm for lumbar spinal applications and 5.5 mm for thoracic spinal applications; maximum flange diameters 68 of 1 to 4 mm greater than maximum shank radii; and anchor sleeve lengths 32 (FIG. 1) appropriate for the intended application. The inventor has determined that the proportions of flange diameter 68 to maximum outer sleeve diameter 128 (i.e., the degree of ‘overhang’ 129 of the flange 24 with respect to the outer circumference of the sleeve 23) may be important in allowing adjacent bone or other body portions to fuse, as for example in a fusion situation with minimal interference from anchor 20. For this purpose, flange overhang dimensions of 2-6 mm will serve in many applications.

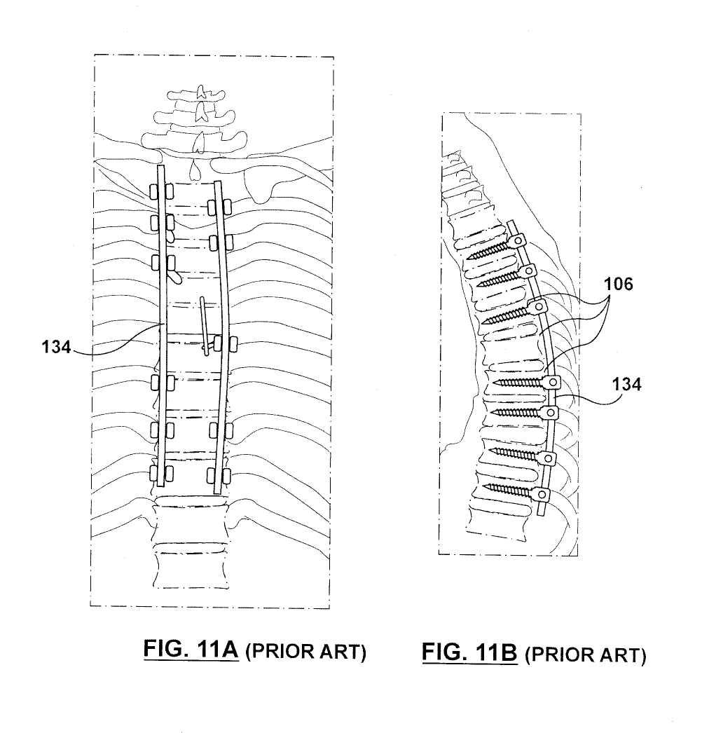

It is to be noted that with some embodiments of the invention, it may be possible to use existing, commercially-available prior art orthopedic screws with anchors suitably-configured according to the invention. Several such screws 22 are shown in FIGS. 9 to 11 without anchors according to the invention. As will be readily appreciated by those skilled in the relevant arts, once they have been made familiar with this disclosure, it may be advantageous in other embodiments to provide screws configured for specific, e.g., optimal, cooperation with anchors according to the invention.

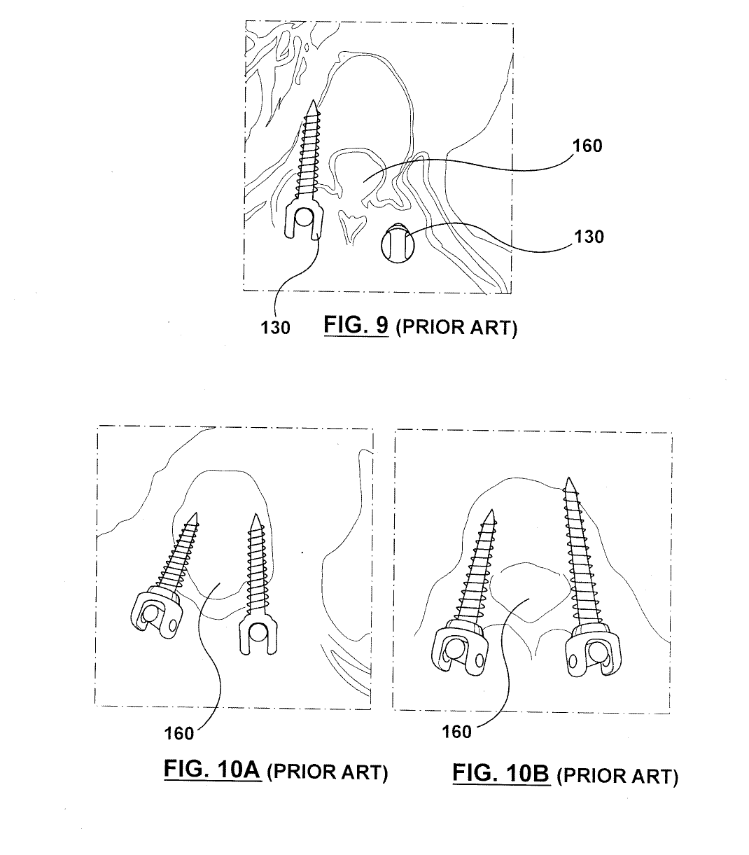

In various embodiments the invention provides methods for posterior placement of spinal implants in living subjects. Such methods include locating at least one pedicle 130 on each of a plurality of vertebrae 106 of an implant subject, as shown in FIG. 6, and establishing in each located pedicle a tract 132 for insertion of a pedicle screw 22; placing a anchor 20 into each established tract 132; installing a pedicle screw 22 into each anchor 20, causing the anchor 20 to expand radially for firmer engagement of the corresponding vertebra 106; and securing a spinal implant to the screw 22.

For example, where it is desired to implant a spinal rod 134 (see for example FIG. 11) by implanting pedicle screws 22 in a plurality of vertebrae 106 of a patient, the desired plurality of vertebrae 106 can be identified, as for example using X-ray devices, probes 136, and/or other tools or procedures, and one or more pedicles 130 or other portions of a posterior aspect 138 can be located within or relative to the identified vertebrae 106, as for example by using a probe 136 such as that shown in FIG. 6. Thereafter a probe 136 or other tool, such as tap 140 or curved pedicle probe 142 can be used to establish a tract 132.

For example, human vertebrae 106 tend to be covered by a relative rigid outer shell, ie, cortical bone 144, with the interior comprising a larger mass of cancellous bone 146. In such cases, once the shell wall 144 has been pierced, as for example using a suitably sharp pointed instrument or a drill, a tract 132 can be established by pushing a probe 136 or other tool 142 into the vertebrae 106 and marrow 146 along the desired trajectory 150, as shown in FIG. 8. For example, depending upon the nature of the structure or device to be implanted, the relative size and strength of the vertebra 106 in which the screw 22 and anchor 20 are to be installed, and the size and length 152 of screw 22 and size and length 32 of the sleeve of anchor 20 to be implanted, an appropriate tract 154, 156 can be established using a straight or curved probe 136, 142, as shown in FIGS. 8A, 8B.

Optionally, tract 132 can be established by using a anchor 20 of suitable length, diameter, and shape; and the anchor 20 may be inserted directly into the corresponding vertebra 106 along the desired trajectory 150 to establish its own tract.

Once a suitable trajectory 150 and/or tract 132 has been established, a probe is placed to confirm that the tract is fully intraosseous. The hole may be tapped using an instrument such as 136. The length of the tract can be estimated by placing an instrument on the probe 132 that is fully seated in the tract, at the point where it meets the start point, so that an appropriate length screw can be chosen. An anchor 20 may be implanted into the corresponding vertebra 106 at this point. For example, as shown in FIG. 7A, a probe 136 comprising a guide pin can be placed into the established tract 132, and a anchor 20 comprising a hole 158 (FIG. 1) placed over the probe or guide pin, and slid into the tract 132 using the guide pin as a guide. When the anchor 20 is emplaced as desired, the probe or guide 136 can be removed, as for example by retracting it, as shown in FIG. 7C. As will be appreciated by those skilled in the relevant arts, such procedures for guiding a anchor 20 into place can be particularly useful where percutaneous or other minimally-invasive procedures are to be used.

As will be appreciated by those skilled in the relevant arts, a particular advantage offered by the invention is the ability to select an anchor and/or screw 22 of length(s) long enough to provide a stable support for screw 22, head 90, and/or any attached implant structures, and not too long, so that the screw 22 and/or anchor 20 extend more deeply into the vertebra than desired, or through the vertebra, as shown in FIGS. 9 and 10B, with attendant risk of piercing a spinal canal 160 or other critical or unsuitable portion of the body of the recipient. For example, the lengths 126, 152 can be relatively short if there is no pedicle deficiency, and are preferably only as long as is necessary to provide adequate support for the contemplated implant, without placing unnecessary mass in body of recipient. As will be understood by those skilled in the relevant arts, once they have been made familiar with this disclosure, the optimum lengths 126, 152 can be determined by use of a probe 136, 408, an X-ray or other image, or other tool.

If necessary, or desired, anchor 20 can be set into the tract, once it has been emplaced, by setting any desired teeth 78 into cortical bone 144 or other relatively rigid portion of the vertebra 106.

With anchor 20 placed in the desired location, and set as desired, a pedicle screw 22 can be placed in the anchor 20 and driven into the anchor 20 by, for example, threading or otherwise placing shank 88 of the screw 22 into passage defined by the sleeve of anchor 20 and using a suitably-configured screwdriver or other driving tool to drive the screw 22 into a desired configuration relative to the anchor 20. Driving of screw 22 into sleeve 23 can cause anchor 20 to expand, by for example causing thread 86 or other portion of screw shank 88 to engage an inner surface 21 of sleeve 23 and push the wall of the sleeve 23 outward radially, as shown by arrows 122. Radial expansion of sleeve 23 can cause the anchor 20 to firmly engage those portions of the vertebra 106 with which it is in contact, and thus stably support the screw 22.

With anchor 20 firmly in contact with the vertebra 106 and screw 22 firmly supported, a spinal implant rod 134 or a plate or other device can be attached to the screw 22 using appropriate attachment means 92 on screw head 90, or other suitable means. The spinal implant rod 134 or plate can be suitably shaped, or otherwise configured, as desired, prior to and/or after attachment to the screw 22.

As will be appreciated by those skilled in the relevant arts, such methods of implanting screws 22 and anchors 20, and other methods disclosed herein, can comprise implanting one or more screw and anchor sets 10 in one or more consecutive vertebrae, and/or skipping one or more vertebrae between implantations, as shown in FIGS. 11A and 11B.

Thus, as will be appreciated by those skilled in the relevant arts, various preferred embodiments of such methods can comprise the percutaneous or other minimally-invasive location of vertebrae and various portions of such vertebrae, including for example, pedicles, and emplacement of screw and anchor sets 10. This is particularly advantageous in installation of pedicle implants and other posterior procedures.

As will be appreciated by those skilled in the relevant arts, lateral and other anterior implantation of screw and anchor sets will frequently require the opening of larger incisions.

As will be further appreciated by those skilled in the relevant arts, the closure of incisions can also be an important part of implantation procedures, particularly where larger incisions are involved, as in many anterior emplacements.

In various embodiments, the invention further provides methods for the lateral or other anterior implantation of screw and anchor sets 10 and other implant devices in living subjects. Such embodiments can comprise exposing anterior aspects 162 of a plurality of vertebrae 106 of the subject (which need not be consecutive or contiguous); establishing at least one tract 164 for a vertebral body screw in each of a plurality of the exposed vertebrae 106; placing an anchor 20 into each established tract; installing a vertebral body screw 22 into each anchor, thereby causing the anchor 20 to expand for firmer engagement of the corresponding vertebra 106; securing a spinal implant such as a rod 134 or plate to the screw; and providing layered closure of the plurality of exposed vertebrae 106.

As with posterior installations as described above, the emplacement of vertebral body screws 40 in lateral or other anterior aspects of vertebrae 106 can comprise the selection of screws 40 of desired lengths 43, and/or of anchors 20 having desired shank lengths 25, and a desired number, length, and configuration of teeth 23, for the emplacement contemplated.

Similarly, in various embodiments of such methods the configuration of implants, such as rods 50 or plates, into one or more desired shapes, can be accomplished either before or after attachment to implanted screws, or both.

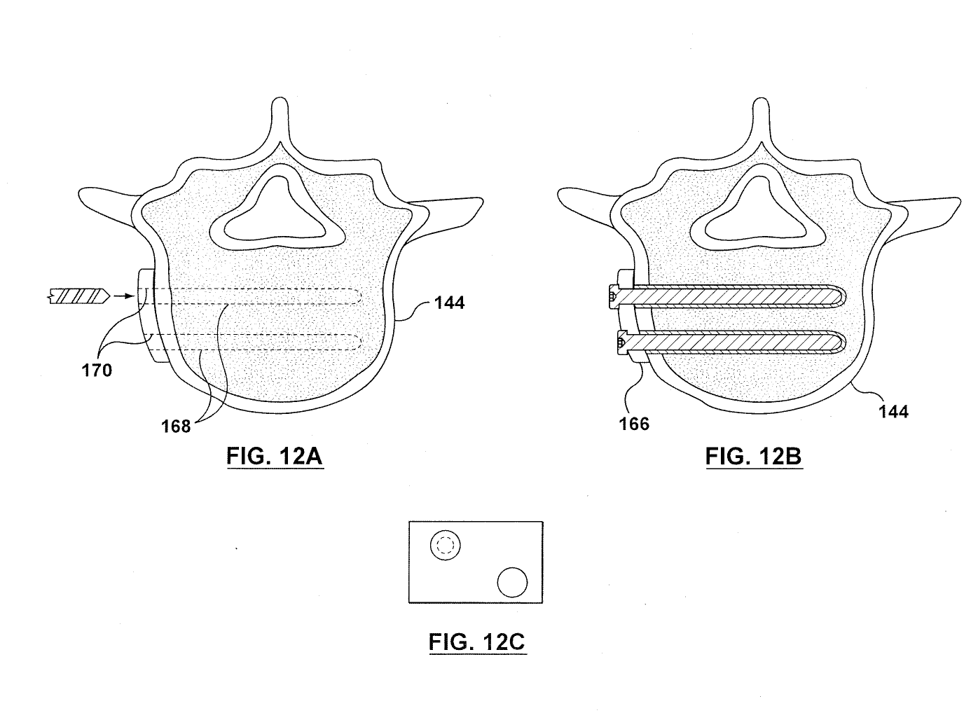

FIG. 12 shows an example of an anterolateral installation of a plate 166 in a vertebra 106. Plate 166 may be placed in a desired position relative to the vertebra(e) 106, and tracts 168 created using an awl or pedicle probe 136, 148. As will be understood by those skilled in the relevant arts, drilling tracts 168 with plate 166 in place can be advantageous, in providing matching dispositions of tracts 168 and holes 170 in plate 166 for passage of screws 22 and anchors 20. With holes 170 and tracts 168 established, anchors 20 may be installed, as herein described. Moreover, the bulk and structural integrity of the implant structure may be optimized by tapping holes 170 in plate 166 to provide threads thereon, and using a plate, rod or other implant structure to support the construct.

It is to be understood that the invention is described herein only in terms of strict medical or surgical possibility. Actual implementation of the invention may be subject to regulatory or other legal or advisory requirement(s), and no representation is made hereby that any particular embodiment complies with any such requirement(s).

While the invention has been described and illustrated in connection with preferred embodiments, many variations and modifications as will be evident to those skilled in the relevant arts may be made without departing from the spirit and scope of the invention, and the invention is thus not to be limited to the precise details of methodology or construction set forth above as such variations and modifications are intended to be included within the scope of the invention. Except to the extent necessary or inherent in the processes themselves, no particular order to steps or stages of methods or processes described in this disclosure, including the Figures, is implied. In many cases the order of process steps may be varied without changing the purpose, effect, or import of the methods described.

1. An orthopedic fixation system, comprising:
an orthopedic screw having a head and a threaded shank thereto;
a screw anchor, the anchor comprising:
a flange comprising a relatively rigid ring having an interior wall defining a generally circular aperture;
a relatively flexible sleeve affixed at a proximal end thereof to the ring, and having an interior wall which together with the aperture of the ring defines a passage for receipt of the shank of the screw therethrough; and wherein:
the distal end of the sleeve has a reduced cross dimension relative to the cross dimension of the shank of the screw when installed therein and the wall of the sleeve has a discontinuity at least the distal end of the sleeve, such that as a leading end of the shank of the screw travels through the passage from the proximal end to the distal end of the sleeve and the shank engages the interior wall of the sleeve the discontinuity permits flexing of the sleeve.

2. The fixation system of claim 6, wherein in installed condition, a base of the screw head and upper surface the ring are in abutment with each other, and the sleeve extends at least 10% of the shank length.

3. (canceled)

4. The fixation system of claim 6, wherein the screw is a pedicle screw and in installed condition, a base of the screw head and upper surface the ring are in abutment with each other, and the sleeve extends between about 10% and 100% of the shank length.

5. (canceled)

6. The fixation system of claim 1, wherein said sleeve wall is provided by metal and said discontinuity is provided by first and second overlapping edges.

7. The fixation system of claim 1, wherein said sleeve wall is provided by a sheet of curved metal having first and second edges which are generally parallel to a central axis of said passage and which overlap with each other both before and after the shank travels through the passage, to provide a wall, when the system is installed in an orthopedic implant, having a continuous outer surface for contact with surrounding bone matter.

8. The fixation system of claim 6, wherein the anchor and screw comprise the same metal, and the metal is stainless steel or titanium.

9. The fixation system of claim 8, wherein the sleeve is of a single piece of sheet metal having a substantially constant thickness of between about 0.002 inches (0.0508 mm) to about 0.006 inches (0.1524 mm).

10. The fixation system of claim 6, wherein the base of the screw head is generally convex and the upper surface of the ring is generally concave such that in installed condition, a distal portion of the head is received into the ring.

11. The fixation system of claim 6, wherein the proximal end of the sleeve is affixed to the ring by welding.

12. (canceled)

13. The fixation system of claim 6, wherein the shank of the screw has a length of between 25 mm and 50 mm, an outer diameter of between about 4.5 mm and 9.5 mm, and the ring aperture is sufficiently large to permit the shank to freely pass therethrough.

14. (canceled)

15. (canceled)

16. The fixation system of claim 13, wherein the ring aperture and screw shank are dimensioned such that, in installed condition, at least 60% of the cross sectional area of the passage through the aperture is occupied by the shank.

17. The fixation system of claim 16, wherein the ring aperture and screw shank are dimensioned such that, in installed condition, the cross sectional area of the passage through the aperture is entirely occupied by the shank.

18. The fixation system of claim 16, wherein the ring aperture and screw shank are dimensioned such that, in installed condition, no more than 90% of the cross sectional area of the passage through the aperture is occupied by the shank.

19. The fixation system of claim 6, wherein the sleeve of the anchor has a length of at least about 10 mm and no more than about 45 mm.

20. (canceled)

21. (canceled)

22. The fixation system of claim 6, wherein in installed condition, the sleeve of the anchor has a length relative to the length of the screw shank such that at least about 10% of the full length of the shank is exposed.

23. The fixation system of claim 22, wherein in installed condition, the sleeve of the anchor has a length relative to the length of the screw shank such that up to about 90% of the full length of the shank is exposed.

24. (canceled)

25. The fixation system of claim 1, wherein the flange further comprises at least one tooth on a distal side of the ring for engaging bone in abutment with the distal side of the ring when the device is installed in a patient.

26. (canceled)

27. The fixation system of claim 25, wherein there are a plurality of said teeth and at least two of the teeth are angularly pointed towards each other such that when the device is installed in a patient with both of the teeth embedded in bone the anchor is fixed against rotation in both the counterclockwise direction and the clockwise direction with respect to a central axis of the aperture.

28. The fixation system of claim 6, wherein the flange further comprises means for fixing the anchor against rotation during installation of the screw.

29. The fixation system of claim 28, wherein the means for fixing the anchor against rotation comprises a depression in a surface of the ring for receipt of a tool therein.

30. The fixation system of claim 28, wherein the means for fixing the anchor against rotation comprises a raised abutment or protrusion on a surface of the flange.

31. An anchor for use in an orthopedic fixation system that includes an orthopedic screw, the anchor comprising:
a flange comprising a relatively rigid ring having an interior wall having a generally circular aperture;
a relatively flexible sleeve affixed at a proximal end thereof to the ring, and having an interior wall which together with the aperture of the flange defines a passage for receipt of the shank of the screw therethrough; and wherein:
the distal end of the sleeve has a reduced cross dimension relative to the cross dimension of the shank of the screw when installed therein and the wall of the sleeve has a discontinuity at least the distal end of the sleeve, such that as a leading end of the shank of the screw travels through the passage from the proximal end to the distal end of the sleeve and the shank engages the interior wall of the sleeve the discontinuity permits outward radial expansion of the sleeve.

32. (canceled)

33. The anchor of claim 54, wherein the sleeve has a length of up to about 50 mm.

34. The anchor of claim 33, wherein the anchor sleeve has a length of about 10 mm.

35. (canceled)

36. (canceled)

37. (canceled)

38. (canceled)

39. (canceled)

40. (canceled)

41. (canceled)

42. (canceled)

43. The anchor of claim 33, wherein the aperture of the flange has a diameter of between about 4.5 mm and 9.5 mm.

44. (canceled)

45. (canceled)

46. (canceled)

47. (canceled)

48. (canceled)

49. (canceled)

50. (canceled)

51. (canceled)

52. The anchor of claim 54, wherein the anchor flange and sleeve are formed of the same metal.

53. (canceled)

54. The anchor of claim 31, wherein said discontinuity is provided by first and second overlapping edges of the wall.

55. The anchor of claim 54, wherein the wall of the sleeve is generally frustroconically shaped, the base of the frustum being located at the proximal end of the sleeve, and the degree of overlap of the overlapping edges of the wall, at the distal end of the sleeve, is between about 10% and about 50%.

56. (canceled)

57. (canceled)

58. (canceled)

59. (canceled)

60. (canceled)

61. A method of manufacture of an anchor for an orthopedic screw, the method comprising:
providing a rigid ring having an interior wall defining a generally circular aperture;
providing a relatively flexible sheet material;
forming the sheet material into a sleeve having a distal end of reduced cross dimension relative to the cross dimension of the aperture of the ring, and having a discontinuity at least the distal end of the sleeve;
affixing a proximal end of the sleeve to ring to form the anchor such that an interior wall of the sleeve together with the aperture of the ring define a passage for receipt of the shank of the screw therethrough; wherein
the sleeve is dimensioned such that as a leading end of the shank of the screw travels through the passage from the proximal end to the distal end of the sleeve the shank engages the interior wall of the sleeve and the discontinuity permits flexing of the sleeve.

62. The method of claim 61, wherein said discontinuity is provided by first and second overlapping edges.

63. A method for installing an orthopedic screw in a patient, the method comprising:
locating a tract in a bone for installation of the screw, and establishing a trajectory therein for the screw;
inserting an anchor into the tract along the established trajectory; and
inserting the screw into the anchor and threading the screw into the tract.

64. The method of claim 63, wherein the anchor comprises a relatively rigid collar at a proximal end thereof and a relatively flexible sleeve wherein the collar has a surface on an underside thereof, and inserting the anchor into the tract includes inserting the sleeve into the tract and bringing the collar into abutting engagement with bone surrounding an opening to the tract.

65. The method of claim 64, further comprising:
selecting a screw having a threaded shank of length and outer diameter suitable for said threading the screw into the tract; and
selecting a sleeve in which the collar has an aperture having an inner diameter which permits insertion of the shank of the selected screw therethrough; wherein,
the sleeve comprises a relatively flexible wall which defines a passage for receipt of the shank therethrough, at least a portion of the wall of the sleeve having an outer diameter smaller than the outer diameter of the shank such that as the screw is threaded into the tract, the leading end of the shank of the screw travels through the passage and engages an interior wall of the sleeve to cause outward radial expansion of the sleeve.

66. The method of claim 65, wherein said wall of the sleeve comprises metal, is generally tubular, and has a discontinuity to permit said outward radial expansion of the sleeve.

67. The method of claim 66, wherein the metal comprises a single sheet of metal rolled into a tube to form the flexible wall such that longitudinal edges of the sheet overlap with each other sufficiently that upon said radial expansion the sleeve completely surrounds the shank of the screw.

68. (canceled)

69. The fixation system of claim 7, wherein in installed condition, a base of the screw head and upper surface the ring are in abutment with each other, and the sleeve extends at least 10% of the shank length.

70. The fixation system of claim 7, wherein the sleeve is of a single piece of sheet metal having a substantially constant thickness of between about 0.002 inches (0.0508 mm) to about 0.006 inches (0.1524 mm).

71. The fixation system of claim 7, wherein the base of the screw head is generally convex and the upper surface of the ring is generally concave such that in installed condition, a distal portion of the head is received into the ring.
